(19) United States
(12) Patent Application Publication
(10) Pub. No.: US 2008/0269317 A1
(43) Pub. Date: Oct. 30, 2008

(54) Compounds for Covalent Binding to MD-2 and Effect on the Immune Response

(76) Inventors: Roman Jerala, Ljubljana (SI); Mateja Mancek Keber, Ljubljana (SI); Robert Bremsak, Kranj (SI)

Correspondence Address: SMITH MOORE LEATHERWOOD LLP, P.O. BOX 21927, GREENSBORO, NC, 27420, US

(21) Appl. No.: 12/065,685

(22) PCT Filed: Aug. 23, 2006
(86) PCT No.: PCT/SI06/00028

§ 371 Date: Apr. 21, 2008

(30) Foreign Application Priority Data
Sep. 6, 2005 (SI) P-200500252

(51) Int. Cl. A61K 31/4015; A61K 39/00; C12N 5/02; A61P 29/00; A61P 37/00

(52) U.S. Cl. 514/425; 424/184.1; 435/375

(57) ABSTRACT

Compounds having a hydrophobic group with a group and capable of reacting with the cysteine residue for the binding to protein MD-2 are disclosed. The compounds are capable of covalently binding to MD-2, which can be either free or in the complex with other molecules. The compounds are capable of replacing other ligands or preventing a binding of other ligands, especially bacterial endotoxin (lipopolysaccharide-LPS), which can otherwise lead towards unwanted activation of the immune response and acute or chronic inflammatory diseases.

### TECHNICAL AREA OF THE INVENTION

The presented invention belongs within the area of pharmaceutical industry and concerns compounds, which bind to MD-2 and inhibit binding of molecules, which are able to activate a MD-2/TLR4 complex of the immune system of human or other higher vertebrates, causing sepsis and other inflammatory diseases. Compounds described in the invention are characterized by containing a chemical group, which covalently binds to the single free cysteine residue of MD-2 and at the same time contains a chemical group responsible for the specific targeting of the compound to the MD-2 binding site and in this way influences the activation of the immune response through the new mechanism of inhibition.

### STATE OF THE ART

Sepsis describes a complex clinical syndrome, which is a result of an excessive and deregulated activation of the host response to an infection. The incidence of sepsis in North America is estimated to 750,000 annually, resulting in 250,000 casualties each year and 200,000 in Europe respectively. Mortality reaches 30%, rising to 40% in the elderly and 50% and greater in patients who develop a septic shock {Angus D C, et al. Crit. Care Med. 2001; 29:13003-1310}. The more exposed group is not only the elderly but also patients with immune deficiency (patients having AIDS, diabetes mellitus, pneumonia) and patients with severe burns and opened wounds. Bacterial sepsis is a cause of septic shock in approximately 60% of cases, out of which Gram-negative bacteria account for about 60%. Sepsis is triggered in the first line by the components of bacterial cell wall, such as lipopolysaccharide (LPS), also known as endotoxin, in Gram-negative bacteria. In this case the disease is called endotoxaemia. According to the progression of symptoms disease states are describes as sepsis, SIRS (systemic inflammatory response syndrome) and septic shock.

Human innate immune system is responsible for the early detection and control of pathogenic microorganisms. Monocytes, macrophages, dendritic cells and some other cells phagocytose pathogenic microorganisms and at the same time coordinate the host response by synthesizing a spectrum of inflammatory mediators and cytokines. Uncontrolled expression of inflammatory cytokines causes immune deregulation, which can lead towards sepsis. Current treatment of sepsis is primarily based on the use of antibiotics and treatment of symptoms. LPS, as the most important initiator of sepsis constitutes the outer membrane of Gram-negative bacteria. It is an amphiphile and temperature stable molecule, consisting of heteropolysaccharides, which are covalently linked to lipid A. Lipid A is the minimal structural fragment, consisting of a diglucosamine backbone, two phosphate groups and four to seven acyl chains. Lipid A is the smallest part sufficient for the endotoxic properties of the molecule and which triggers the cell activation and cytokine synthesis, although other carbohydrate groups potentiate its activity {Rietschel E T, et al. Prog Clin Biol Res. 1987; 231:25-53}, {Haeffner-Cavaillon N, et al. Mol. Immunol. 1989; 26:485}. In the blood stream proteins LBP and CD14 bind LPS {Schumann R R, et al. Science. 1990; 249:1429}, {Wright S D, et al. Science. 1990; 249:1431}. LBP and CD14 are required for the detection of low concentrations of LPS. LPS-LBP-CD14 complex activates the transmembrane protein known as Toll-like protein 4 (TLR4), which through its cytoplasmic TIR domain activates the cascade of reactions, which lead to the activation of a transcriptional factor NF-kappaB and synthesis of TNF-alpha, IL-1, IL-6, IL-8 cytokines. Although TLR4 is indispensable for LPS signaling, the research showed a need for an additional molecule, which is MD-2 {Shimazu R, et al. J Exp Med. 1999; 189:1777}. MD-2 binds to the extracellular domain of TLR4 receptor and binds LPS {Viriyakosol S, et al. J Biol. Chem. 2001; 98:12156-12161}, while TLR4 itself does not bind LPS directly. Mice without MD-2 survive the endotoxic shock. MD-2 is also required for the correct surface expression of TLR4 at the cell surface and for its glycosylation.

Since MD-2 is indispensable for the recognition of LPS and for triggering the cell activation through the synthesis of proinflammatory cytokines, it represents an important therapeutic target for the inhibition of cell activation through LPS, although existing inhibitors of LPS have not been developed on the basis of MD-2 as a target. Inhibitors that prevent LPS binding to MD-2 are therefore able to prevent or stop the development of septic shock.

Other molecules besides LPS that activate cells through the MD-2/TLR4 have been described, such as taxol (paclitaxel), described have been also proteins, which were supposed to activate TLR4, such as HSP70, HSP60, EDA domain of fibronectin, protein of the viral envelope of MMTV . . . . For none of these proteins it has been shown they are able to activate TLR4 without MD-2, which indicates the role of MD-2 in the recognition of those ligands or the presence of LPS contamination.

Known antagonists of LPS are compounds, which structurally resemble LPS by having only single instead of two phosphate groups (MPLA), lower number of acyl chains (4 instead of 6 or 7—e.g. LPS from Rhodobacter sphaeroides or compound 406) or are without of one or both glucosamine groups (AGP). Those compounds were able to inhibit LPS activity. For some of those compounds it was shown they bind to the MD-2/TLR4 complex {Akashi S, et al. J Exp Med. 2003; 198:1035-1042}. Another therapeutic approach for the inhibition of LPS activity represent antibodies against MD-2, LPS or TLR4, where the problem is in the low bioavailability or the need for the parenteral application, low specificity of recognition and fast removal from the bloodstream.

Another notable medical area with the importance of ligand binding to MD-2/TLR4 is the so called “sterile inflammation”, wherein the inflammation is caused by compounds from the organism and not by a bacterial infection. Atherosclerosis belongs to this group of disease, where in human population studies and in research on animal model the importance for TLR4 signaling was shown, although no direct role of MD-2 has been investigated. TLR4 signaling is also involved in the disease progression caused by the environmental pollutants, especially from the atmosphere, such as ozone and ash, produced by oil combustion (ROFA-residual oil fly ash) {Cho H Y, et al. Physiol Genomics. 2005; 22:108-117}. TLR4 is important also in the central nervous system in the pain sensitivity {Tanga F Y, et al. Proc Natl Acad Sci USA. 2005; 102:5856-5861}. In each of the described biological responses of TLR4 the presence of MD-2 was essential and up to now no ligand, with exception of antibodies, which triggers activation has been shown to bind directly to TLR4, but always indirectly mediated through MD-2.

Activation of the system of the innate immunity could have an important role in a prevention of cancer, what has been noticed through the influence of bacterial infection, where the activated monocytes have the main role. The use of bacteria Streptococcus pyrogenes with attenuated virulence activated TLR4 and was shown to work successfully in certain types of tumors. Similar activity was also shown in some LPS mimetics such as e.g. MPLA. It was shown that in patients having tumors, those who expressed TLR4 and MD-2 at the surface of monocytes, responded to the therapy with TLR4 agonists OK-432 and OK-PSA, in 50% causing tumor regression {Okamoto M, et al. J Med. Invest. 2003; 50:9-24}.

### TECHNICAL PROBLEM

Bacterial infection triggers the immune response, which is important for the defense against infection; however the response can progress into the sepsis with very high mortality. The immune response is directly activated by binding LPS to MD-2, which is bound to the TLR4 at the cell surface. The use of antibiotics for treatment of the infection kills microorganisms, but as a result more LPS is released, which additionally stimulates the immune response. Inhibition of LPS binding to MD-2 thus represents the crucial step in prevention of excessive activation of the immune system. Analogously, binding of the ligand to MD-2, which is bound to TLR4, is also characteristic of a number of inflammatory diseases, where no bacterial infection is required and where up to now MD-2 has not yet been used as a target to solve the medical problem. On the other hand a stimulation of the own immune system could be important for the protection of the organism against diseases such as cancer, infection or to increase the efficiency of the vaccination.

Targeted inhibition of MD-2 has not yet been used as an approach to solve the technical problem up to now or compounds have been used which compete with molecules from bacteria and which can be displaced from MD-2 by bacterial molecules.

The aim of the invention is to provide selection and use of compounds for binding to MD-2 with characteristics of compounds being adapted to MD-2 structure in the way they can be used to achieve beneficial effects on health. The advantage of described invention is the fact that binding of compounds to MD-2 is permanent because the covalent bond is formed between the inhibitor and MD-2. Claimed compounds can also be used to treat other inflammatory diseases, where there is no bacterial infection as well as for the stimulation of own immune system.

The typical use of the compounds defined in the invention is as a drug in combination with antimicrobial substances to treat the patients with systemic infection or patients who have a high probability of development of sepsis. Another possible use is as a medicine for patients with chronic inflammatory diseases such as e.g. Cohn's disease, atherosclerosis, inflammation caused by the environmental pollutants, where the use of the compounds defined in the invention can prevent excessive cell activation because of the activation of MD-2/TLR4 complex, therefore in patent claims we define the use of the invention in physiological (diseased) conditions, characterized by the binding of own or foreign ligands to MD-2. Additional use of the invention is for the controlled activation of the immune system through binding to MD-2, where the cell stimulation can increase the efficiency of the vaccination and contributes to the defense the body in treatment of cancer, infection and other diseases, where contribution of the immune system is beneficial.

Detailed description of the invention with examples and Figures/graphs, which show:

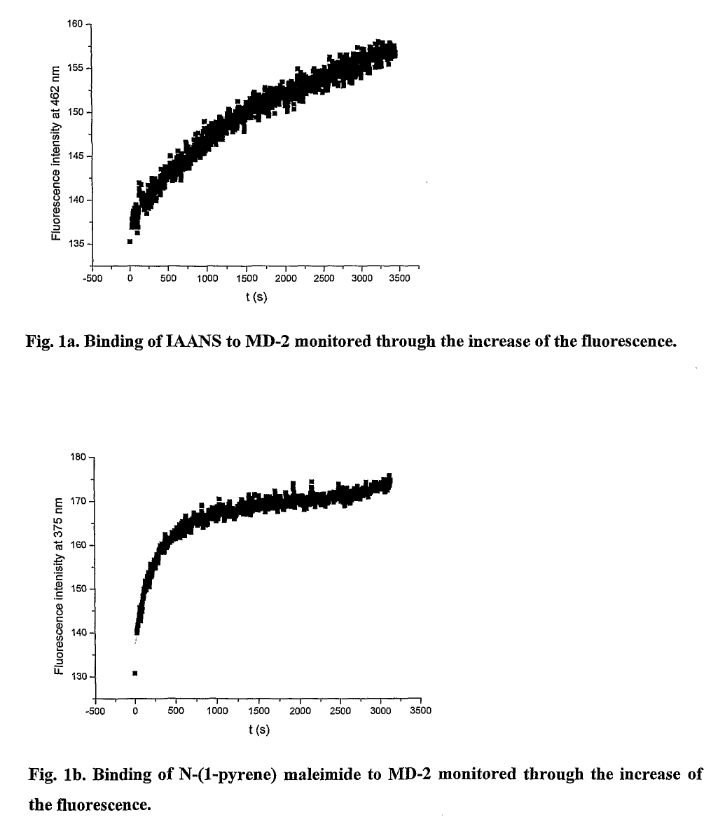

FIG. 1a. Binding of IAANS to MD-2 monitored through the increase of the fluorescence,

FIG. 1b. Binding of N-(1-pyrene)maleimide to MD-2 monitored through the increase of the fluorescence,

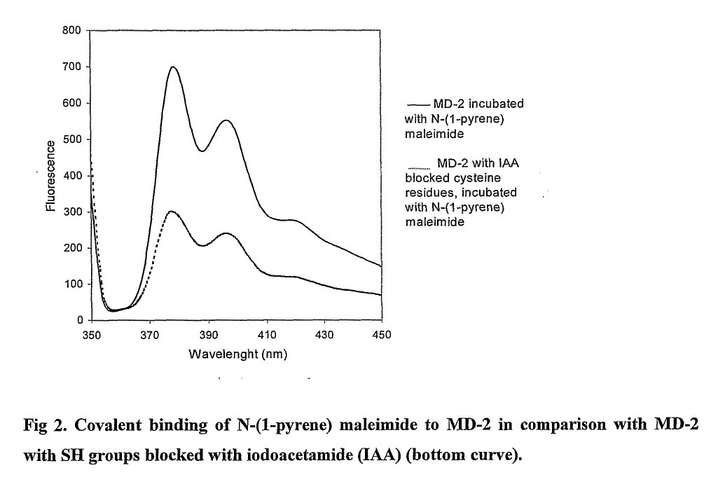

FIG. 2. Covalent binding of N-(1-pyrene)maleimide to MD-2 in comparison to MD-2 with SH groups blocked with iodoacetamide (IAA),

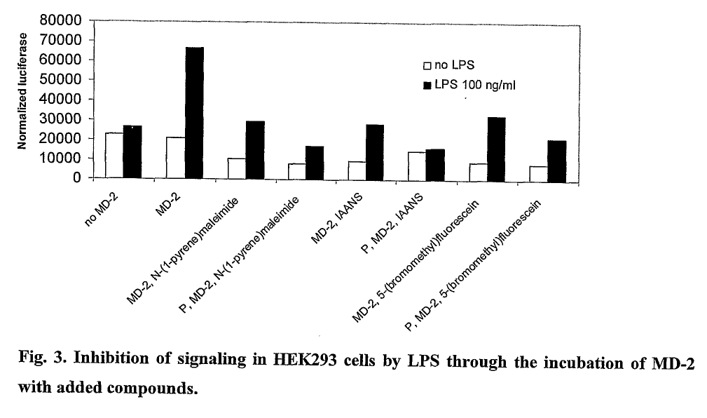

FIG. 3. Inhibition of LPS signaling of HEK293 cells through the incubation of MD-2 with added compounds,

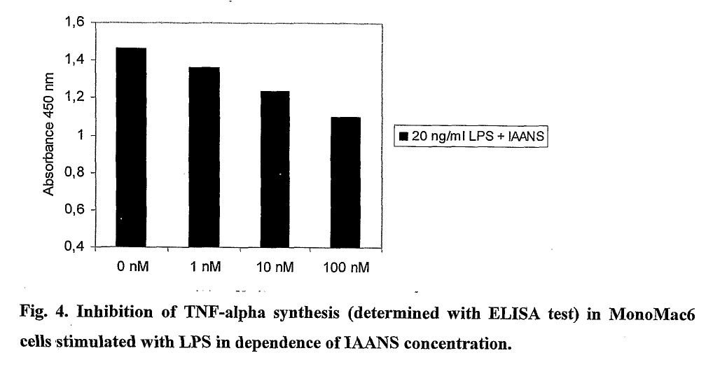

FIG. 4. Inhibition of TNF-alpha synthesis (determined by the ELISA test) in MonoMac6 cells stimulated with LPS in dependence of the IAANS concentration,

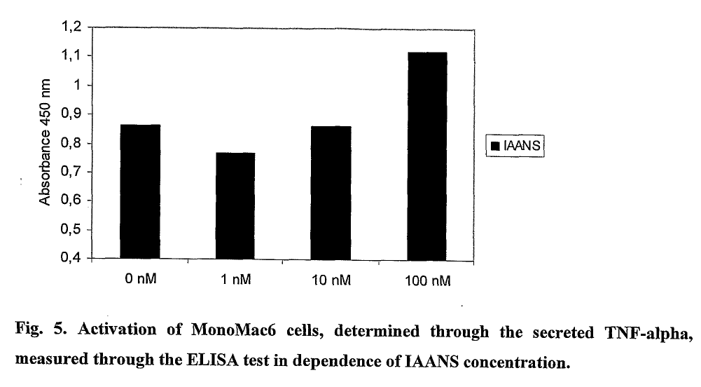

FIG. 5. Activation of MonoMac6 cells, monitored through the secreted TNF-alpha (determined by the ELISA test) in dependence of IAANS concentration.

According to the invention the technical problem is resolved through the combination of two important features of the inhibitors, which we defined based on the three-dimensional model of MD-2 {Gruber A, et al. J Biol. Chem. 2004; 279:28475-82}. We found out that MD-2 contains one free cysteine residue, which lies in close proximity to the LPS-binding site of MD-2 and that MD-2 contains the binding site for hydrophobic compounds. The originality of the invention is the definition of the compounds, which both combines the hydrophobic group, binding to the same hydrophobic binding site on MD-2 as LPS and remains there because the part of the molecule which is reactive with free thiol group covalently binds to the cysteine residue, which is located in the vicinity of this binding site. The advantage brough forth by the invention is that the compound remains covalently bound to MD-2 and inhibits LPS binding, which would otherwise activate the signaling and activation of the cells of the immune system. Activation of the immune system through LPS activates the signaling cascade, which leads to the translocation of the protein NF-kappaB into the nucleus, where it activates the transcription of a number of genes of proinflammatory proteins, most prominent among which is TNF-alpha, so we use the activation of NF-kappaB responsive genes or TNF-alpha secretion as a marker of the cell activation and key step in the development of the inflammatory diseases. The functional group, which reacts with free cysteine residue of MD-2, can a person skilled in the art choose among different groups, which form a covalent bond with thiol (SH) group of cysteine residue such as, but without limitations, thiol, disulfide, alkylhalide, maleimide group, organic-mercury compounds, nitrosyl thiols, and thioesters. Hydrophobic group, which binds to the LPS-binding site on MD-2 can be selected without limitations from the groups containing one or more, preferably between one and six, alkyl or acyl chains, condensed aromatic and heterocyclic rings, preferably one to five rings, anilino-naphthalene-sulfonic group, pyrene and other groups, which preferably contain from four to fifty carbon atoms. It is beneficial if the compound, which binds to LPS-binding site on MD-2, contains one or more anionic groups, which persons skilled in the art can select without limitations from the groups such as phosphate, sulphate, carboxyl group, because the interaction of anionic groups with basic residues of MD-2 increases the stability and selectivity of binding.

Antagonists of LPS or MD-2/TLR4 described up to now are in equilibrium between free and MD-2 bound form, in most cases have a strong tendency to aggregate and are quickly removed from the bloodstream. Compounds of this invention with described features and applications can be used directly and person skilled in the art can choose the modifications of their precursors from numerous modifications, which enable metabolic conversion in organism to the final form, reactive with MD-2, modifications selected but without limitations from the groups such as thioesters, different mixed disulfides, thioeters, thiosulphonates such as methylmetanthiosulphonate, 2,2-dimethylpropanthioat. The compounds have a biological effect on all cells expressing TLR4 and can be used as a medicine in the concentration range between 0.1 nM to 1 mM. The preferable use of binding of compounds to MD-2 is for the preparation of medicines for diseases, where TLR4 and/or MD-2 play important role in the development of the disease, mainly in inflammatory diseases including acute inflammation such as microbial infection and sepsis or chronic inflammation such as atherosclerosis, Cohn's disease, and inflammation caused by the environmental pollutants.

The first example of our invention demonstrates the binding capability of the compounds, which contain a hydrophobic group and a group, which reacts with cysteine residue, to the MD-2 protein.

The second example of the invention demonstrates the irreversible (covalent) binding of said compounds to MD-2.

The third example of the invention demonstrates that binding of said compounds to MD-2 inhibits the activation of LPS signaling pathway and secretion of inflammatory cytokines, particularly, but not only, TNF-alpha.

The fourth example of the invention demonstrates that binding of said compounds to MD-2 triggers weak activation of the cells of the human immune system without the addition of LPS or its derivates.

In the first example of the invention we used recombinant MD-2 produced in bacteria Escherichia coli to test the binding of the compounds, which contain a hydrophobic group and a group, which binds to the cysteine residue. This protein has biological activity as endogenous MD-2 and added protein enables response to LPS to cells lacking endogenous MD-2. We tested binding of compounds 2-(4′-(iodoacetamido)anilino)naphthalene-6-sulfonic acid (IAANS) and N-(1-pyrene)maleimide, which in addition to the above mentioned properties also have the additional feature of fluorescence, which makes them useful to show the principle of the invention. The use of both compounds is suitable, as it enables simple detection of binding to MD-2. Change of the environment, which is a result of binding of the compound into the hydrophobic pocket, causes an increase of the fluorescence intensity of compounds, which is detected by measuring the emission fluorescence spectra using the spectrofluorimeter.

In the second example of the invention we show, that binding of the compound to MD-2 is irreversible, therefore after binding to MD-2, compound remains permanently bound to the protein and therefore prevent binding of LPS or other ligands to MD-2.

With the third example of the invention we show that binding of said compounds to MD-2 inhibits the activation of human cells by LPS and therefore we indicate the application of these compounds for the treatment of sepsis and other inflammatory diseases, where own or foreign ligand binds to MD-2 and causes the cell activation through TLR4. One of the prominent uses of compounds is for the prevention of an exaggerated immune response because of stimulation with bacterial LPS, which activates the cells by binding to MD-2/TLR4. Expected applications include the prophylactic use on humans and animals, in cases of high probability for the development of sepsis or other inflammatory diseases as well as for the treatment of the progressed diseases, where the said compounds could displace the ligand from MD-2.

With the fourth example of the invention we show that binding of said compounds to MD-2 can weakly activate the cells of the human immune system without the addition of LPS. Therefore said compounds could activate the system of the innate immunity, which can be significant for the prevention of cancer development and its treatment, stimulation of the immune response at vaccination and for the treatment of viral infections.

We explain the invention, but do not restrict it with the following examples. Anywhere in examples, where the reagents are not specified they are of the quality needed for work in molecular biology and biochemistry.

### EXAMPLE 1

Binding of inhibitors, which contain the thiol reactive, hydrophobic and anionic groups, was performed on spectrofluorimeter LS55 (Perkin Elmer, GB). We used 1 ml quartz cuvette (optical length 10.0×5.0 mm, Hellma Suprasil, Germany). All measurements were performed at 25° C.

IAANS (Molecular Probes, USA) is an example of a compound with an alkylhalide group, which is reactive with a thiol group, an anilinonaphthalene group as a hydrophobic group and a sulfonic group as an anionic group. N-(1-pyrene) maleimide (Molecular Probes, USA) contains a maleimide group as a thiol reactive group and a pyrene as a hydrophobic group. Both compounds were dissolved in DMSO. IAANS concentration was determined by measuring the absorbance at 326 nm in methanol (extinction coefficient at 326 nm in methanol is 27 000 cm⁻¹M⁻¹). Concentration of N-(1-pyrene) maleimide was determined by measuring the absorbance at 338 nm in methanol (extinction coefficient at 338 nm in methanol is 40,000 cm⁻¹M⁻¹). Binding to MD-2 increases the fluorescence intensities of both compounds. Time dependent binding to MD-2 was measured by following the change of emission maximum after the addition of 100 nM MD-2 to 200 nM compound dissolved in milliQ water (IAANS ext/emis 326/462 nm, N-(1-pyrene)maleimide 338/375 nm). The results are on FIG. 1a and FIG. 1b.

FIG. 1a represents binding of IAANS to MD-2. To 200 nM IAANS 100 nM MD-2 was added. We measured the change of the fluorescence intensity at emission maximum of IAANS. The curve represents the increase of the IAANS fluorescence after binding to MD-2.

FIG. 1b represents binding of N-(1-pyrene)maleimide to MD-2. To 200 nM IAANS 100 nM MD-2 was added. We measured the change of the intensity at emission maximum of N-(1-pyrene) maleimide. The curve represents the increase of the N-(1-pyrene) maleimide fluorescence after binding to MD-2.

### EXAMPLE 2

The compounds with features mentioned above covalently bind to MD-2. The binding experiment was performed by adding to the concentration of MD-2 two times concentration excess of N-(1-pyrene) maleimide and incubated one hour at room temperature in the dark. The compound, which nonspecifically (noncovalently) bound to MD-2 was released from the protein by denaturation of the protein in 6 M guanidinium hydrochloride (GdnHCl). To the dissolved denatured protein we added five volumes of chilled (−20° C.) acetone and incubated for additional 60 min at −20° C. Then we centrifuged for 10 min at 13 000 rpm. Protein with covalently bound compound was precipitated with acetone, while the unbound compound remained dissolved in the solution. Acetone was removed and the precipitate was additionally washed with acetone, centrifuged and acetone removed. Remaining acetone was removed by drying of the precipitate at room temperature. The precipitate was dissolved in 200 g of 6 M GdnHCl and fluorescence emission spectrum was measured with excitation at 338 nm (FIG. 2). Covalent binding of compound to MD-2 was verified by previous incubation of five molar excess of iodoacetamide, which covalently binds to free cysteine residue of MD-2 and blocks them to the large extent. Binding of iodoacetamide inhibited binding of a compound to free cysteine residue on MD-2, which can be detected by lower fluorescence intensity of the compound.

FIG. 2 shows covalent binding of N-(1-pyrene) maleimide to MD-2. We compared the fluorescence of MD-2, incubated with N-(1-pyrene) maleimide and to MD-2, with said compound, where we have previously added iodoacetamide for blocking free cysteine residues. MD-2 dissolved in 6 M GdnHCl preserved the emission spectrum of N-(1-pyrene) maleimide, showing that the compound irreversibly bound to MD-2. Since the addition of iodoacetamide inhibited binding of N-(1-pyrene) maleimide to MD-2 this confirms that the compound binds to free cysteine residues of MD-2.

### EXAMPLE 3

Effect of compounds on biological activity of MD-2 for the activation of human cells by LPS was measured by the reporter luciferase assay. This system provides the information of the activation of the LPS signaling pathway, which represents the first stage in the activation of the inflammatory response, induced by LPS or other compounds. Reporter firefly luciferase is an enzyme, whose expression in the cell is due to the coupling with promotor linked to the cell activation. In our case a promotor was added, which binds the transcription factor NF-kappaB, which is activated by the addition of LPS in cells, which contain or to which we have added TLR4 and MD-2. The amount of synthesized luciferase depends on the degree of cell activation and can be quantitatively determined from the amount of the emitted light after the addition of the substrate for the luciferase. The efficiency of the transfection and the number of cells can not be optimized, so the results are normalized by using additional reporter system using Renilla luciferase, whose expression in cells is independent from cell activation. The amount of Renilla luciferase is determined by measurement of the emitted light, similar as in the case of firefly luciferase.

For the experiments we have used human HE 93 cell line, which does not express proteins MD-2 and TLR4. One day before the transfection we seeded 5×10⁴ cells per well into the 96-well plate containing DMEM medium (Invitrogen, San Diego, USA) with 10% FBS (BioWittaker, Walkersville, Md., USA). The cells were transfected with TLR4 plasmid and reporter plasmids, NF-kappaB dependent firefly luciferase and Renilla luciferase. Per well we prepared a transfection mixture according to the manufacturer instructions: 0.5 μl lipofectamine (Invitrogen), 50 ng of TLR4 plasmid, 80 ng of firefly luciferase plasmid and 5 ng of Renilla luciferase reporter plasmid. After 4 hours we exchanged media with 100 μl of DMEM with 2% FBS. 24 hours after the transfection we performed two variants of the test:

a). We added 50 nM of recombinant MD-2 to the cells, incubated for 1 hour and added 100 nM of the compounds tested, incubated for 1 hour and added 100 ng/ml LPS. After 24 hours we lysed the cells and determined the amount of the reporter proteins using the “Dual-luciferase reporter assay system” (Promega, Madison, Wis., USA) on microplate reader Mithras LB 940 (Berthold Technologies, Germany). The data were analyzed as shown on FIG. 3.
b). Recombinant MD-2 and the compounds were incubated together for 1 hour and then 50 nM of MD-2 was added to cells and incubated for an additional hour before the addition of 100 ng/ml LPS. After 24 hours we lysed the cells and determined the amount of the reporter proteins with “Dual-luciferase reporter assay system”, results were normalized and shown in FIG. 3.

FIG. 3 shows the inhibition of LPS signaling. To HEK293 cells, which express TLR4, we added MD-2 in two ways: a). MD-2 was added to the cells and then we added the compounds and LPS. b). MD-2 and the compounds were preincubated and the mixture was added to the cells followed by LPS (marked by a letter P). After 24 hours we measured the luciferase activity and normalized the results. Inhibition of LPS activation because of the addition of the compounds is noticeable, which is even more pronounced if MD-2 and the compounds were preincubated.

In the example we show the tested compounds IAANS, N-(1-pyrene) maleimide and 5-(bromomethyl) fluorescein, although persons skilled in the art can choose other compounds, which have the features described in the invention. If the compounds have inhibitory effect on the activation of LPS signaling pathway the synthesis of luciferase will be lower, so the amount of the released light will be low after the addition of the substrate.

The effect of certain compound on the cell activation by LPS can also be detected by determination of the amount of cytokines, which are released by the cell activation. Addition of LPS to macrophage cells (or other cells, which normally express MD-2 and TLR4) causes the secretion of inflammatory cytokines such as TNF-alpha, IL-1, IL-8 . . . If the compound inhibits cell activation, cells release lower amount of cytokines into the medium. The amount of cytokines released into the media can be determined by the ELISA test. For the experiment we used monocytic cells MonoMac6. To 96-well plate we seeded 1×10⁵ cells per well in media for MonoMac6 cells (RPMI with 10% FBS, nonessential aminoacids and OPI (both from Sigma)). 50 ng/ml of phorbol 12-myristate 3-acetate (Sigma) needed for differentiation into macrophages was added to cells. IAANS was added at different concentrations and incubated 1 hour, followed by the addition of 20 ng/ml LPS. 20 hours after the stimulation we took the supernatant, centrifuged it for 3 min on 13 000 rpm and performed the ELISA test for TNF-alpha. The test was performed according to the manufacturer's instructions (Immuno Tools, Germany) as are shown in FIG. 4.

FIG. 4 shows the inhibition of TNF-alpha synthesis by IAANS. To MonoMac6 cells, which express MD-2 and TLR4, we added different concentrations of IAANS and LPS and determined the concentration of TNF-alpha in media using the ELISA test. The addition of IAANS inhibited LPS dependent synthesis of TNF-alpha in a concentration-dependent manner.

The inhibition of cell activation is the first step in prevention of an exaggerated immune response at acute inflammatory diseases, such as, but not exclusively, microbial infections, sepsis or chronic inflammatory diseases, such as, but not exclusively, Cohn's disease, atherosclerosis or inflammation caused by the environmental pollutants. The concentrations of the compounds, which can be used for the preparation of a medicine for the prevention of the diseases, characterized by the ligand binding to MD-2 are preferable in the range between 1 ng/ml to 5 mg/ml. Besides the compounds, which bind to MD-2, additional substances can be added to the drugs, such as inert compounds or compounds with different activity, such as, but not exclusively, antimicrobial or anti-inflammatory activity.

### EXAMPLE 4

The activation of the host innate immune system can have an important role for the treatment of diseases such as microbial infections, cancer and other diseases, where the participation of the innate immunity system is important. It is important that the compound, which activates the innate immune system has no other effects. If the compound activates the cells of the innate immunity, the same cytokines are secreted as by the activation with LPS, so the same assay can be used. For the assay we have used human monocytic cells MonoMac6. Into the 96-well plate we seeded 1×10⁵ cells per well in the media for MonoMac6 cells. We added 50 ng/ml of phorbol 12-myristate 3-acetate (Sigma), which is needed for differentiation of cells into macrophages. Then we added different concentrations of IAANS and incubated. 20 hours after the stimulation we took the cell supernatants, centrifuged for 3 min at 13 000 rpm and performed ELISA test for TNF-alpha. The test was performed according to the manufacturer's instructions (Immuno Tools, Germany) and shown in FIG. 5.

FIG. 5 shows the activation of MonoMac6 cells with IAANS. To MonoMac6 cells we added different concentrations of IAANS. IAANS activated the cells in the concentration dependent manner. Cells secreted TNF-alpha, which stimulates the innate immune response, which is important for the acquired immunity at vaccination or treatment of disease such as cancer, where the activation of host immune response is desirable. Persons skilled in the art can choose compounds with features, described in the invention, therefore by selecting a functional group, reactive with free thiol group of MD-2 and that contains a hydrophobic group, which mostly consists of nonpolar heavy atoms and linear, branched, cyclic groups or their combinations and preferably contains from three to fifty carbon atoms. The concentration of compounds, which is used for the preparation of a medicine for the activation of the immune system, is preferably in the range between 1 ng/ml to 2 mg/ml.

1-22. (canceled)

23. A compound for reducing TLR4-induced inflammatory response, the compound comprising:
(a) a hydrophobic group; and
(b) a group capable of interacting with a cysteine residue of MD-2,
wherein the compound is configured to reduce the binding of a ligand to the TLR4, thereby reducing inflammatory response.

24. The compound according to claim 23, wherein the group capable of interacting with a cysteine residue is configured to interact with a free cysteine residue.

25. The compound according to claim 23, wherein the compound is configured to reduce the binding of LPS to TLR4.

26. The compound according to claim 23, wherein the group capable of interacting with a cysteine residue of MD-2 is selected to form a covalent bond with the cysteine residue

27. The compound according to claim 26, wherein the group capable of interacting with a cysteine residue of MD-2 is selected to form a covalent bond with a thiol (SH) group of the cysteine residue.

28. The compound according to claim 23, wherein the group capable of interacting with a cysteine residue includes a functional group selected from a thiol, a disulfide, an alkylhalide, a maleimide group, an organic-mercury compound, a nitrosyl thiol, a thioester, and mixtures thereof.

29. The compound according to claim 23, wherein the hydrophobic group includes a functional group selected from a one to six member alkyl chain, a one to six member acyl chain, an aromatic ring, a heterocyclic ring, an anilino-naphthalene-sulfonic group, a pyrene, and mixtures thereof.

30. The compound according to claim 23, wherein the compound further includes an anionic group.

31. The compound according to claim 30, wherein the anionic group includes a functional group selected from a phosphate, a sulphate, a carboxyl group, and mixtures thereof, thereby increasing the stability and selectivity of compound binding.

32. A compound for reducing inflammatory response, the compound comprising:
(a) a hydrophobic group including a functional group selected from a one to six member alkyl chain, a one to six member acyl chain, an aromatic ring, a heterocyclic ring, an anilino-naphthalene-sulfonic group, a pyrene, and mixtures thereof,
(b) a group configured to form a covalent bond with a free cysteine residue of MD-2, the group including a functional group selected from a thiol, a disulfide, an alkylhalide, a maleimide group, an organic-mercury compound, a nitrosyl thiol, a thioester, and mixtures thereof, and
(c) an anionic group including a functional group selected from a phosphate, a sulphate, a carboxyl group, and mixtures thereof, thereby increasing the stability and selectivity of compound binding,
wherein the compound is configured to block the binding of a ligand to a TLR4/MD-2 complex, thereby reducing inflammatory response.

33. A drug combination for reducing excessive activation of the immune system in response to bacterial infection, the combination comprising:
(a) an antimicrobial substance in an amount sufficient to kill microorganisms, whereby killing releases LPS from the microorganisms; and
(b) about 0.1 nM to about 1 mM of a second compound, the second compound comprising:
(i) a hydrophobic group, and
(ii) a group capable of interacting with a cysteine residue of MD-2,
wherein the second compound is configured to block the LPS from binding to TLR4, thereby reducing excessive immune system response.

34. The combination according to claim 33, wherein the group capable of interacting with a cysteine residue is configured to interact with a free cysteine residue.

35. A method of treating inflammation in a patient comprising administering a compound configured to bind to a cysteine residue of MD-2.

36. The method according to claim 35, wherein the inflammation is bacterial-induced inflammation.

37. The method according to claim 35, wherein the inflammation is auto-induced inflammation.

38. The method according to claim 35, wherein the inflammation is environmentally-induced inflammation.

39. The method according to claim 35, wherein the inflammation is induced by sepsis or endotoxaemia.

40. The method according to claim 35, further including identifying a patient suffering from inflammation.

41. The method according to claim 40, wherein the identifying includes identifying a patient suffering from sepsis or endotoxaemia.

42. The method according to claim 35, wherein the compound is configured to covalently bond to a free cysteine residue.

43. The method according to claim 35, wherein the compound comprises:
(a) a hydrophobic group; and
(b) a group capable of interacting with a cysteine residue of MD-2,
wherein the compound is configured to reduce the binding of a ligand to TLR4, thereby reducing inflammatory response.

44. The method according to claim 43, wherein the group capable of interacting with a cysteine residue includes a functional group selected from a thiol, a disulfide, an alkylhalide, a maleimide group, an organic-mercury compound, a nitrosyl thiol, a thioester, and mixtures thereof.

45. The method according to claim 43, wherein the hydrophobic group includes a functional group selected from a one to six member alkyl chain, a one to six member acyl chain, an aromatic ring, a heterocyclic ring, an anilino-naphthalene-sulfonic group, a pyrene, and mixtures thereof.

46. The method according to claim 43, wherein:
(a) the hydrophobic group includes a functional group selected from a one to six member alkyl chain, a one to six member acyl chain, an aromatic ring, a heterocyclic ring, an anilino-naphthalene-sulfonic group, a pyrene, and mixtures thereof, and
(b) the group capable of interacting with a cysteine residue of MD-2 includes a functional group selected from a thiol, a disulfide, an alkylhalide, a maleimide group, an organic-mercury compound, a nitrosyl thiol, a thioester, and mixtures thereof, and
(c) the compound further includes an anionic group including a functional group selected from a phosphate, a sulphate, a carboxyl group, and mixtures thereof, thereby increasing the stability and selectivity of compound binding.

47. The method according to claim 35, wherein the compound is selected from the group consisting of 2-(4′-(iodoacetamido)anilino) naphthalene-6-sulfonic acid (IAANS)N-(1-pyrene)maleimide, and mixtures thereof.

48. The method according to claim 36, further including administering an antimicrobial agent.

49. The method according to claim 35, wherein the compound is not selected from the group consisting of MPLA, compound 406, anti-MD-2 antibody, anti-LPS antibody, and anti-TLR4 antibody.

50. A method of inhibiting TLR4 signaling, the method comprising:
(a) obtaining a cell that does not express MD-2 and TLR4;
(b) transfecting the cell with DNA encoding a TLR4 receptor;
(c) contacting the cell with a MD-2;
(d) contacting the cell with a ligand that binds to TLR4; and
(e) contacting the cell with a compound comprising
(i) hydrophobic group; and
(ii) a group capable of interacting with a cysteine residue of MD-2.

51. The method according to claim 50, wherein the group capable of interacting with a cysteine residue is configured to interact with a free cysteine residue.

52. The method according to claim 50, wherein the group capable of interacting with a cysteine residue includes a functional group selected from a thiol, a disulfide, an alkylhalide, a maleimide group, an organic-mercury compound, a nitrosyl thiol, a thioester, and mixtures thereof.

53. The method according to claim 50, wherein the hydrophobic group includes a functional group selected from a one to six member alkyl chain, a one to six member acyl chain, an aromatic ring, a heterocyclic ring, an anilino-naphthalene-sulfonic group, a pyrene, and mixtures thereof.

54. The method according to claim 50, wherein the ligand includes LPS.

55. A method of eliciting an immune response in an animal, the method comprising:
testing the animal for disease; and
introducing a compound into the animal comprising a group capable of interacting with a cysteine residue of MD-2.

56. A method of eliciting an immune response in an animal, the method comprising:
introducing an antigen into the animal; and
introducing a compound comprising a group capable of interacting with a cysteine residue of MD-2.
